(19) United States
(12) Patent Application Publication
(10) Pub. No.: US 2021/0179507 A1
(43) Pub. Date: Jun. 17, 2021

(54) PROCESS FOR BIOLOGICAL AMMONIA PRODUCTION BY NITROGEN FIXING CYANOBACTERIA

(71) Applicant: GROUPONICS GREENHOUSE TECHNOLOGY LTD., Kiryat Bialik (IL)

(72) Inventors: Lior HESSEL, Kiryat Bialik (IL); Assaf SHEMESH, Haifa (IL)

(21) Appl. No.: 17/272,315

(22) PCT Filed: Aug. 29, 2019
(86) PCT No.: PCT/IB2019/057284

§ 371 Date: Feb. 28, 2021

Related U.S. Application Data
(60) Provisional application No. 62/724,457, filed on Aug. 29, 2018.

(51) Int. Cl. C05C 3/00; C05G 5/23; C12N 1/20; C05F 11/08

(52) U.S. Cl. CPC C05C 3/00; C05F 11/08; C12N 1/20; C05G 5/23

(57) ABSTRACT

Disclosed are methods and systems for the production of ammonia biologically using a strain of nitrogen fixing bacteria of the Nostocaceae family.

### CROSS-REFERENCES TO RELATED APPLICATIONS

This application is related to and claims priority from commonly owned U.S. Provisional Patent Applications: Ser. No. 62/724,457, entitled: PROCESS FOR BIOLOGICAL AMMONIA PRODUCTION BY NITROGEN FIXING CYANOBACTERIA, filed on Aug. 29, 2018, the disclosure of which is incorporated by reference in its entirety herein.

### TECHNICAL FIELD

The present invention is directed to methods and systems for producing ammonia with nitrogen fixing cyanobacteria.

### BACKGROUND

Nitrogen fertilizer in conventional agriculture is provided almost exclusively via the Haber Bosch process. This process takes a heavy toll on global energy production and natural gas resources. In organic agriculture, where sustainability is a main factor, nitrogen from a biological or a naturally occurring source is needed.

Organic farmers employ crop rotation with legumes, or distribute animal manure in order to enrich the soil with nitrogen compounds. However, modern techniques such as fertigation and hydroponics require water soluble fertilizers that keep organic matter to a minimum. Organic fertilizers on the market today have several disadvantages, which render them impractical for robust production. These fertilizers are composed of substances such as blood meal and fish bone, and rely extensively on Sodium Nitrate, which causes soil salinization (and high salinity in water based growth). The Nitrogen in these fertilizers is mainly in the form of inaccessible peptides and amino acids, which only become available after a long period of time. Some of these fertilizers contain solid residues, which clog the irrigation piping. In a hydroponic high-yield setting, these fertilizers tend to behave poorly by being unstable in the long term, and causing drastic changes in water parameters such as conductivity and pH. As a result, most hydroponic growers use chemical fertilizers such as Calcium Nitrate and Potassium Nitrate.

Nitrogen fixing cyanobacteria have been actively investigated since the 1980s. As discussed in, Bothe, Hermann, et al, “Nitrogen fixation and hydrogen metabolism in cyanobacteria,” in Microbiology and Molecular Biology Reviews, 74, No. 4 (2010), pp. 529-551, Nitrogen fixing cyanobacteria produce an enzyme called nitrogenase that can fix nitrogen from the air and convert it to ammonia. This enzyme must be surrounded by low ammonia and oxygen levels in order to function effectively. The nitrogen fixation occurs in specialized cells of the cyanobacteria called heterocysts, which provide ammonia to the vegetative cells and receive photosynthesis derived sugar in return. As discussed in Musgrave, Stephan C., et al., “Sustained ammonia production by immobilized filaments of the nitrogen-fixing cyanobacterium Anabaena 27893,” in Biotech Letters, Vol. 4, No. 10 (1982), pp. 647-652, blocking the ammonia assimilation pathway by applying an inhibitor to the enzyme Glutamine synthetase (e.g. L-Methionine Sulfoximine or Methionine Sulfoximine (MSX)) releases the ammonia to the medium.

### SUMMARY

The present invention encompasses a process for the production of ammonia biologically using a strain of Nitrogen fixing bacteria, a cyanobacteria of the Nostocaceae family. This Nitrogen fixing cyanobacteria converts ammonia to Nitrogen for use, for example, as a fertilizer.

The bacteria are grown in a tank, such as a raceway tank or continuous photobioreactor (the terms “tank”, “photobioreactor”, “bioreactor”, and “reactor”, are used interchangeably herein), which maintains optimal growing conditions for the cells. Nitrogen, carbon dioxide, and/or air are supplied as gas, and other minerals are incorporated in the medium. The bacteria perform photosynthesis and fix nitrogen into ammonium ions that are released to the medium. The medium is continuously separated from the cells and transferred to a nitrification unit to produce a Nitrate rich solution suitable for use as an organic hydroponic fertilizer. A water treatment unit is used to concentrate the solution and return excess water to the tank. Liquid in the tank (photobioreactor) is circulated with an ample interface with the gas phase to provide aeration to the cells. Nitrogen and carbon dioxide are metabolized by the cyanobacteria and excess oxygen is stripped away.

The invention provides for biological nitrogen fixation into ammonia by cyanobacteria of the family Nostocaceae, such as Anabaena sp., in a growing tank or bioreactor at a high yield continuous process. For example, the cyanobacteria are maintained viable at a constant density, or their density is kept at repeating cycles. This can be achieved, for example, by maintaining a constant and sufficiently low ratio between the concentration of ammonium uptake inhibitor and the cyanobacteria cells, which induce ammonia excretion without killing the cells. Ammonia is produced at a constant rate or at a repeating cycle rate depending on cell density, lighting level or other parameters. The ammonia is constantly removed from the photo-bioreactor to the nitrification unit where it is continuously converted to nitrate and so on. This process may go on for an extended time period such as weeks, months or years. This is in contrast to batch processes which occur for limited time periods, typically less than a week. The vast majority of the art describes batch processes in which cyanobacteria are induced to produce ammonia over a short time period. At the end of these processes, the cyanobacteria lose their viability and have to be replaced.

The invention provides for the extraction of ammonia from the growing tank or bioreactor for use as a raw product, such as a fertilizer in agriculture.

The invention provides for the continuous conversion of ammonia into other forms of fixed nitrogen, such as Nitrate, for use as a raw product, such as a fertilizer in agriculture.

The invention provides for the continuous concentration of ammonia or other type of fixed nitrogen species for use as a concentrated raw product.

The invention provides for a continuous process for the growth of ammonium producing, nitrogen fixing cyanobacteria in a growth tank. The cyanobacteria is grown in suspension, or immobilized on carriers. The carriers are, for example, foams, fibers or any material, which are capable of holding the cyanobacteria in place.

The invention provides for a continuous fertilization of crops with the nitrate rich product of the cyanobacteria system.

Systems of the invention use a raceway tank or bioreactor to grow cyanobacteria, and circulation is achieved using a paddle wheel. A water pump or an airlift pump is used to drive fresh medium into the main tank. Excess medium, rich in ammonium ions, leaves through the liquid outlets to the nitrification unit that may or may not contain a trickle (or trickling) filter.

Systems of the invention use a wet-dry setting. Cyanobacteria are immobilized on carriers, fibers or foams laid on top of a mesh or a screen above the water level. Fresh medium is sprayed or trickled over the carriers, and the medium that drips from the carriers is rich in ammonium ions, and continues to the nitrification unit.

Systems of the invention include a closed tubular reactor (photobioreactors or bioreactor) to grow the cyanobacteria with a lower contamination risk. The cells are grown on carriers or in suspension in transparent tubes, and are separated, if necessary, before the medium is transferred to the nitrification unit.

Systems of the invention are such that a main tank is covered and sealed. A gas mixture is sparged through the inlet ports into the main tank or bioreactor. Excess gas, rich in ammonia, leaves through the gas outlet ports, and is introduced to the nitrification unit by bubbling or another method.

Systems of the invention use closed flat panel airlifts to grow cyanobacteria. The gas supply is sparged from the bottom and provides aeration and agitation in the panels. Ammonia rich gas in the headspace flows out to the nitrification unit.

The invention is such that Nitrate rich solutions from the nitrification unit may or may not be concentrated using reverse osmosis units, or other concentration systems and methods.

Systems of the invention use a controller such as a computer to regulate the continuous fertilization of crops with the nitrate rich product of the cyanobacteria system. This product may be concentrated or dilute.

Embodiments of the invention are directed to a method for producing ammonia. The method comprises: growing nitrogen fixing cyanobacteria in a bioreactor; exposing the cyanobacteria, while in a viable state, to an inhibitor, in the bioreactor, such that the inhibitor induces the cyanobacteria to release ammonia; and, preserving the cyanobacteria in the viable state for continuously producing the ammonia.

Optionally, the method additionally comprises: providing media to the bioreactor, and the media for receiving the released ammonia.

Optionally, the method is such that the viable state includes a live state.

Optionally, the method is such that the ammonia includes at least one of ammonia, ammonium ions, or, a mixture of ammonia and ammonium ions.

Optionally, the method is such that the it additionally comprises: controlling the pH level in the bioreactor to alter the balance of ammonia to ammonium ions.

Optionally, the method is such that the media is aerated with a gas stream prior to being provided to the bioreactor.

Optionally, the method is such that the bioreactor includes liquid solution.

Optionally, the method is such that the it additionally comprises: agitating the liquid solution in the bioreactor.

Optionally, the method is such that the cyanobacteria is grown in suspension.

Optionally, the method is such that the cyanobacteria is immobilized on one or more carriers.

Optionally, the method is such that the carriers include one or more of foams, fibers or any material, which is capable of holding the cyanobacteria in place.

Optionally, the method is such that the carriers include one or more of: alginate or carrageenan beads, polyvinyl, polyester, or polyurethane foams, polyester fibers, cellulosic or poly-sulfone hollow fibers, or, clay particles.

Optionally, the method is such that the clay particles comprise one or more of silica, alumina, combinations thereof, or composites thereof.

Optionally, the method is such that the cyanobacteria is from the family Nostocaceae.

Optionally, the method is such that the family Nostocaceae includes the genus Anabaena.

Optionally, the method is such that the genus Anabaena comprises the species: A. flos aqua, A. siamensis, A. azollae, A. variabilis, or mutant strains thereof.

Optionally, the method is such that the media includes at least one of: BG-11, a blue green algae media, or a nitrogen-free blue green algae media.

Optionally, the method is such that the gas stream includes one or more of: Nitrogen, Carbon Dioxide or Air.

Optionally, the method is such that the bioreactor includes a tank.

Optionally, the method is such that the bioreactor includes at least one tube which is at least translucent.

Optionally, the method is such that the tank includes a sparger.

Optionally, the method is such that the bioreactor includes at least one flat panel airlift reactor.

Optionally, the method is such that the at least one flat panel airlift reactor includes a sparger.

Optionally, the method is such that the bioreactor includes a sparger.

Optionally, the method is such that the ammonia includes ammonia gas dissolved in the liquid solution as a mixture of soluble ammonia gas and ammonium ions.

Optionally, the method is such that the ammonia gas dissolved in the liquid solution is exposed to nitrifying bacteria to produce a Nitrate based product.

Optionally, the method is such that the ammonia gas is exposed to nitrifying bacteria to produce a Nitrate based product.

Optionally, the method is such that the Nitrate based product includes fertilizer.

Optionally, the method is such that the Nitrate based product includes liquid fertilizer.

Optionally, the method is such that the cyanobacteria is grown at an alkaline pH.

Optionally, the method is such that the pH is approximately 9 to 10.

Optionally, the method is such that the it additionally comprises: continuously aerating the bioreactor to force ammonia out of the bioreactor.

Optionally, the method is such that the exposing to nitrifying bacteria includes passing the ammonia gas dissolved in the liquid through a biofilter.

Optionally, the method is such that the biofilter includes one or more of: polypropylene bio balls, ceramic porous blocks, polyester fibers and activated carbon.

Optionally, the method is such that the exposing to nitrifying bacteria includes bubbling the ammonia gas into a biofilter.

Optionally, the method is such that the biofilter, into which the ammonia\ gas is bubbled into, includes one or more of: polypropylene bio balls, ceramic porous blocks, polyester fibers and activated carbon.

Optionally, the method is such that the inhibitor includes at least one of: MSX (L-methionine-DL-sulfoximine), MSO (L-methionine-sulfone), phosphinothricin ((RS)-2-Amino-4-(hydroxy(methyl)phosphonoyl)butanoic acid), or, Bialaphos (L-Alanyl-L-alanyl-phosphinothricin) or Glyphosate (N-(phosphonomethyl)glycine).

Optionally, the method is such that the inhibitor is provided to the bioreactor with the media.

Embodiments of the invention are directed to a method for producing ammonia. The method comprises: growing nitrogen fixing cyanobacteria in a bioreactor, wherein the cyanobacteria is a mutant strain of cyanobacteria; controlling the environment in the bioreactor, such that the cyanobacteria, while in a viable state, releases ammonia; preserving the cyanobacteria in the viable state for continuously producing the ammonia; and, extracting the ammonia from the bioreactor including separating the ammonia from the cyanobacteria and the inhibitor.

Optionally, the method is such that the viable state includes a live state.

Optionally, the method is such that the controlling the environment includes controlling one or more of agitation, temperature, and pH in the bioreactor.

Optionally, the method is such that the mutant strain of cyanobacteria includes at least one of: A. variabilis, or, A. siamensis.

Embodiments of the invention are directed to a method for producing ammonia. The method comprises: growing nitrogen fixing cyanobacteria in a bioreactor; exposing the cyanobacteria, while in a viable state, to an inhibitor, in the bioreactor, such that the inhibitor induces the cyanobacteria to release ammonia; preserving the cyanobacteria in the viable state for continuously producing the ammonia; and, extracting the ammonia from the bioreactor including separating the ammonia from the cyanobacteria and the inhibitor.

Optionally, the method is such that the ammonia is in at least one of a liquid phase, or a gas phase.

Embodiments of the invention are directed to a method for producing ammonia. The method comprises: growing nitrogen fixing cyanobacteria in a bioreactor; exposing the cyanobacteria, while in a viable state, to an inhibitor, in the bioreactor, such that the inhibitor induces the cyanobacteria to release ammonia; preserving the cyanobacteria in the viable state for continuously producing the ammonia; and, exposing the ammonia to nitrifying bacteria to produce a Nitrate based product.

Optionally, the method is such that the Nitrate based product includes fertilizer.

Optionally, the method is such that it additionally comprises: providing the fertilizer to a hydroponic unit for vegetation.

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components. In the drawings:

FIG. 1A is diagram of a side view of a first embodiment of an open system for performing processes in accordance with the present invention;

FIG. 1B is diagram of a top view of a first embodiment of the system for performing processes in accordance with the present invention;

FIG. 1C is diagram of a showing the system of FIG. 1A in greater detail;

FIG. 1D is a side view of a second embodiment of the growing system;

FIG. 1E is a side view of a third embodiment of the growing system;

FIG. 2A is diagram of a side view of a fourth embodiment of a closed system for performing processes in accordance with the present invention;

FIG. 2B is diagram of a top view of a fourth embodiment of the system for performing processes in accordance with the present invention;

FIG. 2C is diagram of a showing the system of FIG. 2A in greater detail.

FIG. 2D is a side view of a fifth embodiment of a closed growing system;

FIG. 3A is a diagram of a smart fertilization setting based on the nitrate rich product of the cyanobacteria system; and,

FIG. 3B is a diagram of another smart fertilization setting based on the nitrate rich product of the cyanobacteria system.

### DETAILED DESCRIPTION OF THE DRAWINGS

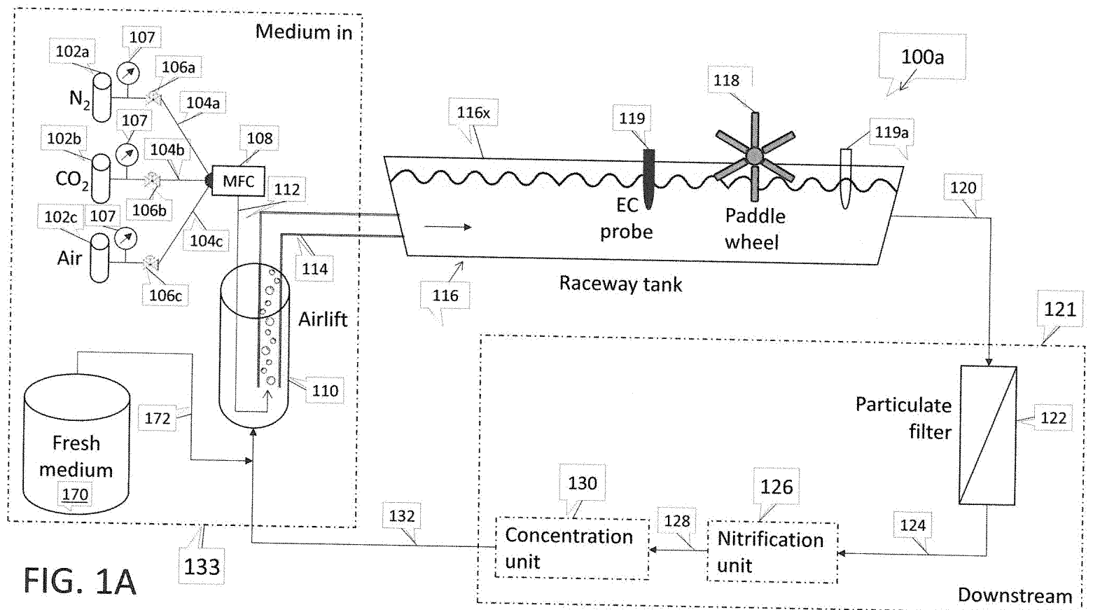

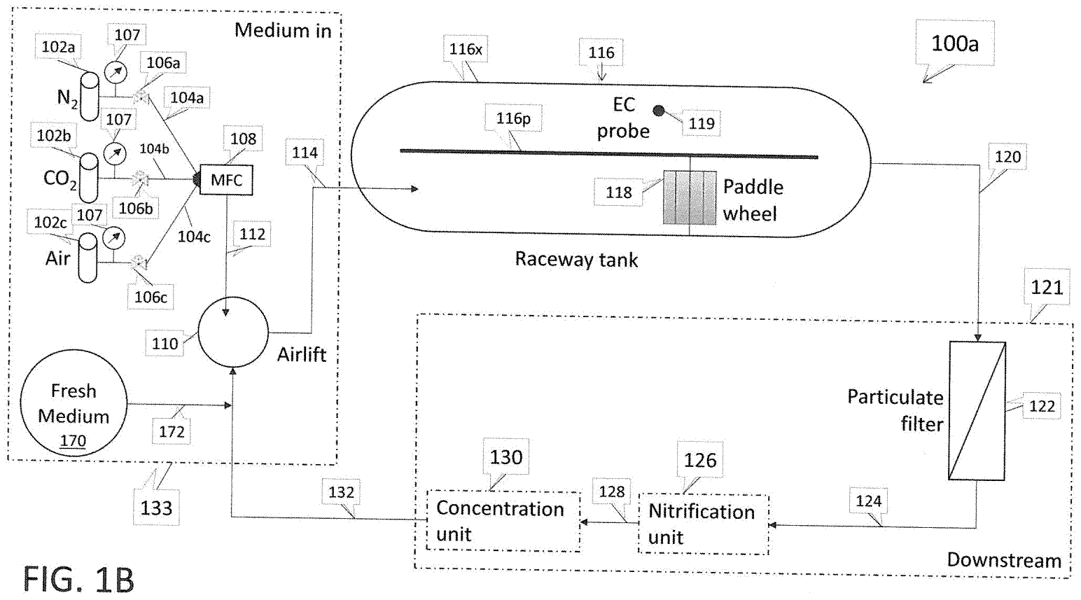

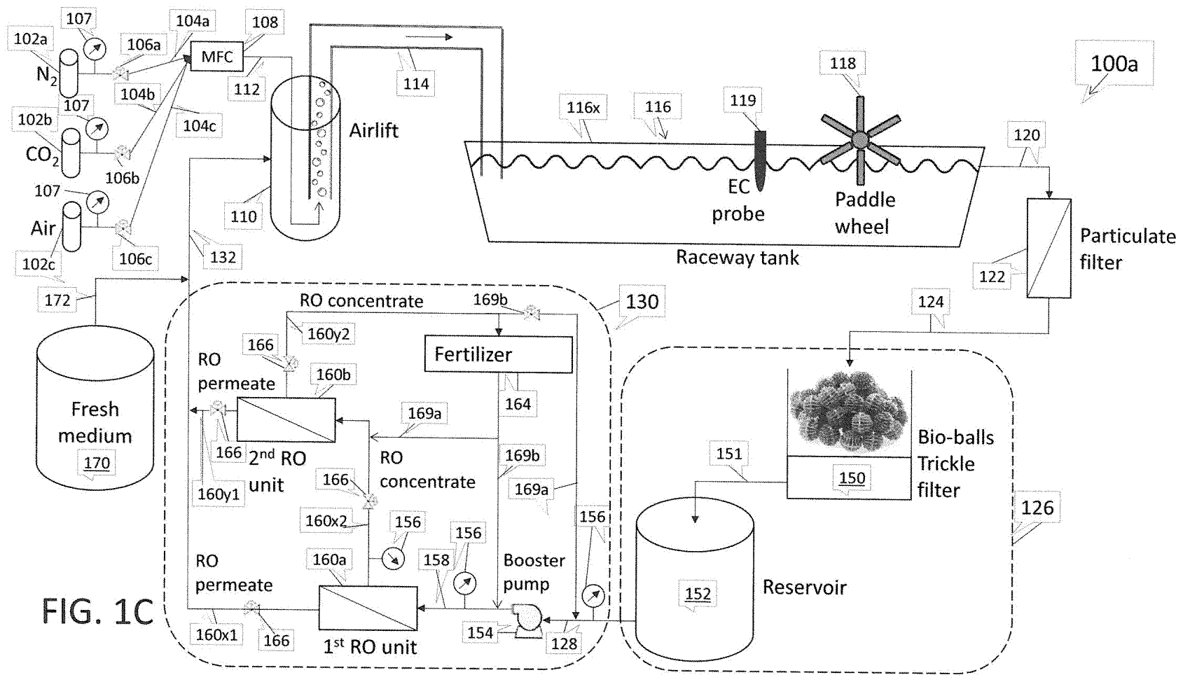

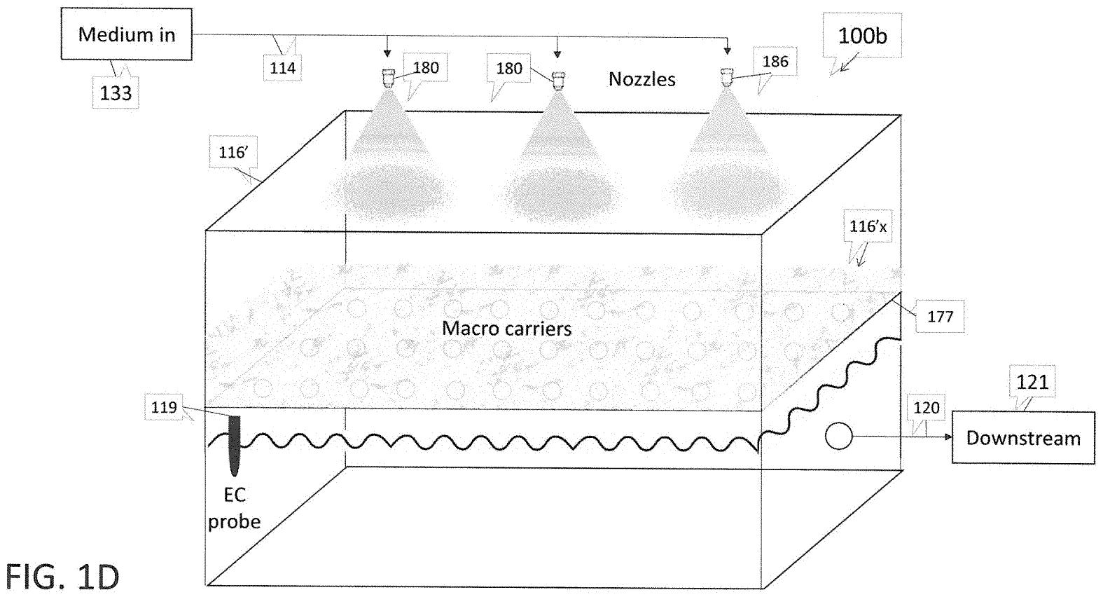

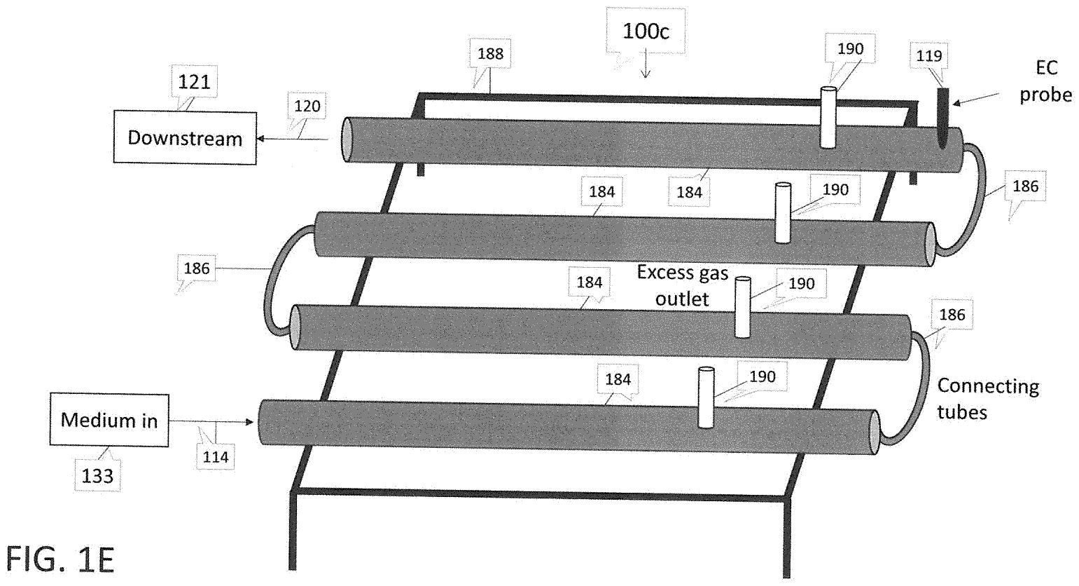

FIGS. 1A and 1B provide a system 100a for performing a process in accordance with an embodiment of the invention. Sources of Nitrogen (N₂) gas 102a, Carbon Dioxide (CO₂) gas 102b and air 102c connect over lines 104a, 104b, 104c (with valves 106a, 106b, 106c), with one or more of the Nitrogen, Carbon Dioxide, or Air forming a gas stream. The gas stream is provided to a mass flow controller (MFC) 108 or a similar apparatus, which adjusts the flow rate of each gas. As used herein, “lines” include conduits, tubes, carriers, and the like between structures, through which fluids, e.g., liquids and/or gasses, move or travel. Each of the lines 104a-104c includes, for example, a pressure gauge 107, which is optional. An airlift pump 110 receives gas, i.e., the gas stream, from the MFC 108, which controls the gas influx into the airlift pump 110. The gas is received by the airlift pump 110 from the MFC 108 over a line 112. The airlift pump 110 mixes the gas with water, and forces the mixed gas/water through lines 114 into a tank, photobioreactor or bioreactor 116 (the terms “tank”, “photobioreactor”, “bioreactor”, and “reactor” are used interchangeably herein in this document). The tank or bioreactor 116 is sealed with a cover 116x, or the like (to maintain pressure therein and keep gasses from escaping) and provides a controlled environment for growing and maintaining cyanobacteria (e.g., nitrogen fixing cyanobacteria) in a viable, e.g., live, state. While one airlift pump 110 is shown, multiple airlift pumps 110 may also be used.

The airlift pump 110 uses compressed gas to drive and aerate fresh medium (in a media feed) from a medium tank 170 into the tank 116. The airlift pump 110 also functions to continuously aerate the tank (bioreactor) 116 to force ammonia out of the tank (bioreactor), through the outlet line 120. This aeration by the airlift pump 110 supplements the water lost to evaporation and nutrients consumed by the cyanobacteria (in the tank 116, as detailed below). The fresh medium, for example, includes purified water, and a nutrient solution that is added by an automatic control system according to the sampled conditions in the tank 116. The concentration of these nutrients is correlated to electrical conductivity (EC), which is measured, for example, by an electrical conductivity (EC) probe 119. While an EC probe 119 is shown, other probes, electrodes and sensors for example, for measuring temperature, pH, dissolved oxygen, ammonia, nitrate, CO₂, ion conductivity, oxidation reduction potential, or other process parameter, may also be used to monitor tank 116 conditions. In addition, optional turbidity sensors 119a may be applied to measure cell density. An example of a turbidity sensor 119a is a Hamilton Dencytee sensor.

Additionally, one or more of the aforementioned process parameters, for example, may be regulated using the proper intervention in the tank 116, such as acid/base pumps, temperature control units, gases flow rates, circulation rate or the like.

The tank 116 typically holds cyanobacteria, and accordingly, operates as a bioreactor. The tank 116 includes a paddle wheel 118 or other agitator for the water. The cyanobacteria is, for example, grown in suspension or is immobilized on carriers inside the tank 116a. For example, Nitrogen fixing cyanobacteria, namely the family Nostocaceae, is grown in the tank 116, in order to produce a high nitrogen liquid fertilizer. Examples of cyanobacteria species (e.g., from the family Nostocaceae) include members of the genus Anabaena, comprising species such as A. flos aqua, A. siamensis, A. azollae, A. variabilis, or mutant strains. When the cyanobacteria is grown in suspension in the tank, the cyanobacteria may form films or aggregates. When immobilized on carriers, the carriers are, for example, alginate or carrageenan beads, polyvinyl, polyester, or polyurethane foams, polyester fibers, cellulosic or poly-sulfone hollow fibers, clay particles (e.g., from clay minerals) composed of elements such as silica or alumina, or combinations or composites of such materials. These are divided into micro-carriers with a typical size of hundreds of micro meters (μm), which keep the cells in suspension in an agitated solution, and macro-carriers that are large enough to be visible with the naked eye, and allow the separation of the cells from the medium by a simple mesh or a strainer.

The tank 116 is, for example, a D-ended raceway tank, which is shallow, typically a few decimeters deep, for example, approximately 25 cm deep, with a partition 116p (FIG. 1B) in the middle, to encourage laminar water circulation. The paddle wheel 118, which is optional, is submerged approximately half way into the depth of the tank 116, and by rotating the paddles, the medium is circulated around the tank 116. This circulation facilitates gas exchange. Other circulation devices or circulators may also be used in place of the paddle wheel, should it be desired.

The tank 116, via a line (outlet line) 120 connects to a processing unit 121 (shown by the broken line box), which includes a filtration unit 122, nitrification unit 126, and a concentration unit 130. The filtration unit 122 includes, for example, a particulate filter. The filtration unit 122, via a line 124, connects to the nitrification unit 126, which through a line 128, connects to the concentration unit 130. A line 132 connects the concentration unit 130 to the airlift pump 110.

The components 102a-102c, 108, 110, 116 and 121 (filtration unit 122, nitrification unit 126 and concentration unit 130) (and 170) are arranged as a circuit. This circuit arrangement provides for the continuous production of nitrogen, for example, as fertilizer.

FIG. 1C shows the system 100a in detail, on which an example operation is now described. Initially, cyanobacteria has been grown in suspension or on carriers in the tank 116 and has released ammonium ions (NH₄⁺) into the water. This is due to the cyanobacteria fixing nitrogen, causing it to release (excrete) ammonia, the ammonia including ammonium ion (ammonium), ammonia, ammonium ions and ammonia in a mixture, into the water (or liquid including aqueous solution) of the tank or photobioreactor 116. The cyanobacteria is typically induced to release the ammonium or ammonia by adding inhibitors to enzymes in their ammonium uptake pathways, such as MSX (L-methionine-DL-sulfoximine) or MSO (L-methionine-sulfone), phosphinothricin ((RS)-2-Amino-4-(hydroxy(methyl)phosphonoyl)butanoic acid), Bialaphos (L-Alanyl-L-alanyl-phosphinothricin) or Glyphosate (N-(phosphonomethyl)glycine), or a brand formulation of these substances such as Roundup (Bayer, Germany), for example, in the media feed, in the tank 116, or both. However, certain mutant strains of cyanobacteria, such as: A. variabilis SA-1 (Spiller, H., et al. “Isolation and characterization of nitrogenase-derepressed mutant strains of cyanobacterium Anabaena variabilis.” Journal of bacteriology 165.2 (1986): 412-419), A. variabilis ED81 and ED92 (Kerby, Nigel W., et al. “Photoproduction of ammonium by immobilized mutant strains of Anabaena variabilis.” Applied microbiology and biotechnology 24.1 (1986): 42-46), A. siamensis SS1 (Thomas, Selwin P., Arieh Zaritsky, and Sammy Boussiba. “Ammonium excretion by an L-methionine-DL-sulfoximine-resistant mutant of the rice field cyanobacterium Anabaena siamensis.” Appl. Environ. Microbiol. 56.11 (1990): 3499-3504), and, A. variabilis PCC 7937-C9 (Bui, Lan Anh, et al. “Isolation, improvement and characterization of an ammonium excreting mutant strain of the heterocytous cyanobacterium, Anabaena variabilis PCC 7937.” Biochemical engineering journal 90 (2014): 279-285), typically do not require an inhibitor, to release ammonia.

Nitrogen, Carbon Dioxide, and/or Air, from sources 102a-102c, respectively, form a gas stream, which is injected through the MFC 108, into the airlift pump 110. The airlift pump 110 forces the gas stream and the aerated medium (e.g., BG-11 media, such as Gibco® BG-11₀ media from Thermo Fisher Scientific, a Blue Green Algae Media, or a nitrogen-free blue green algae media, stored in the storage tank 170) to flow into the tank 116, which is filled with liquid cyanobacteria suspended in the medium. The cyanobacteria is viable (e.g., in a viable state), being able to survive, multiply and live successfully in an active state, including, for example, being able to release ammonia, after exposure to an inhibitor of one or more of its ammonia uptake pathway, such as MSX (e.g., once circulated in the bioreactor 116). Circulation is achieved with the paddle wheel 118, a submersible pump or a similar method. Fixed nitrogen in the form of ammonium ions (produced by the cyanobacteria in suspension or associated with carriers) is dissolved in the liquid solution medium. A relatively low pH level (e.g. pH 7) ensures that the balance between ammonia and ammonium ions, shifts towards the ammonium, and therefore the ammonia vapor pressure is kept to a negligible level. The ammonium ions are dissolved in a liquid solution, and processed in a liquid phase, and also for systems 100b, 100c (detailed below). Throughout the process the cyanobacteria is preserved or otherwise kept or maintained so as to be viable, in the aforementioned viable state (e.g., live state), for a prolonged time period (e.g., weeks, months or years), in order that ammonia is continuously produced (by a continuous process).

For example, the cyanobacteria are maintained viable at a constant density, or their density is kept at repeating cycles. This can be achieved, for example, by maintaining a constant and sufficiently low ratio between the concentration of ammonium uptake inhibitor and the cyanobacteria cells, which is, for example, at 1.5 μmol MSX/mg chlorophyll. This induces ammonia excretion without killing the cells Ammonia is produced at a constant rate or at a repeating cycle rate depending on cell density, lighting level or other parameters. The ammonia produced is removed from the tank (photobioreactor) 116 to the nitrification unit 126 where it is continuously converted to nitrate. This process may go on for an extended time period such as weeks, months or years.

Excess solution in the tank 116, typically rich in ammonium ions, overflow the liquid outlets and passes, over an outlet line 120 from the tank 116, to the filtration unit 122, and its particulate filter, to remove detritus and avoid clogging in the system 100a. The flow rate of the solution, as it flows through the tank 116 and filtration unit 122, is set by the pumping rate of the airlift pump 110. The pumping rate is, for example, a rate permitting power saving, but not where any nitrogen fixation is hindered by a high ammonium concentration in the medium.

The filtered solution, from the filtration unit 122 is then passed, for example, by being bubbled into the nitrification unit 126. The nitrification unit 126 includes, for example, a bio-filter or substrate 150, for example, a trickle filter, and a reservoir 152, connected to a line 151. The bio-filter 150 includes filtration media of bio balls, which support colonies of nitrifying bacteria (from genera such as Nitrosomonas and Nitrobacter), to convert ammonium ions NH₄⁺ to Nitrite (NO₂), then to Nitrate (NO₃⁻). The bio balls are, for example, polypropylene, and may be, for example, Tetra BB Bio balls by Tetra Holdings GmbH (Germany) or BioMate filter media by Lifeguard Aquatics (USA). Other suitable filtration media include, ceramic porous blocks, polyester fibers and activated carbon. Additionally, the bio-filter may be made of a porous or high surface area cationic media such as coral gravel, aragonite, calcite beads, or crushed magnesite. The cationic media, include, for example, carbonates or alkalis of calcium, magnesium or potassium.

The solution of ammonium ions is passed through the bio balls media in the bio-filter 150, where the ammonium ions are converted to Nitrate (NO₃⁻) ions. The Nitrate rich solution is then received in the reservoir 152. The booster pump 154, through line 128, receives the Nitrate rich solution and forces the Nitrate into the concentration unit 130. Pressure gauges 156, which are optional, are, for example, placed along the line 128 as well as the other lines 160x1, 160x2, 160y1, 160y2 of the concentration unit 130.

The concentration unit 130, in addition to the booster pump 154, includes reverse osmosis (RO) units, for example two RO units 160a, 160b (with RO filters), arranged sequentially, and a reservoir 164 for the concentrate from the sequentially arranged filters 160a, 160b. These RO units 160a, 160b serve to concentrate the solution of ammonium ions. Alternately, the RO units 160a, 160b, are arranged in parallel.

The booster pump 154 pumps the liquid Nitrate filtrate through the RO units 160a, 160b at a rate sufficient to separate the accumulated Nitrate rich solution into concentrate. Initially, the booster pump 154 pumps the Nitrate rich solution into the first RO unit 160a, via line 158. The permeate from the RO unit 160a is sent along line 160x1 which continues into line 132 to the airlift pump 110. The concentrate from the RO unit 160a is sent along line 160x2 to the second RO unit 160b. The permeate from the second RO Unit 160b is sent along line 160y1 which continues into line 132 to the airlift pump 110. The concentrate from the second RO Unit 160b is sent along line 160y2 to the reservoir 164, so as to be recovered as product, e.g., fertilizer (liquid fertilizer). Optionally, some of the concentrate 160y2 may be returned to the RO unit 160a, 160b, via a line 169, which is controlled by valve 169b, for additional RO filtration in order to achieve a higher final concentration. The permeate from the RO filters 160a, 160b is enriched with fresh medium, e.g., BG-11₀ (from Thermo Fisher Scientific, or self-prepared) or other medium containing minerals, buffers and other elements required by cyanobacteria, from a fresh medium source 170, e.g., a tank, and redirected over lines 172 and 132 to the tank 116, via the airlift pump 110, as detailed above.

In the concentration unit 130 the lines 160x1, 160x2, 160y1 and 160y2 include valves 166. These valves 166, along with the valves 106a-106c, MFC 108, airlift pump 110, paddle wheel 118, EC probe 119, and booster pump 154, of the system 100a, may be controlled manually, automatically by a computer control system, or combinations thereof. Also, the pressure gauges 107, 156 may also be connected to the computer control system.

FIG. 1D shows an alternate system 100b with a tank (photobioreactor or photoreactor) 116′. The tank 116′ includes a mesh screen (or cover) 116x′, made of polypropylene or Polymethyl Methacryclate (PMMA) with drilled holes, for example. On top of the screen 116x, above the water level, carriers 177 are placed and are inoculated with cyanobacteria. Fresh medium arriving from a line 114 is injected into the tank 116′ by nozzles 180, connected to the line 114, or other drip apparatus, to irrigate the carriers 177, which are located above the water level. This setting, commonly referred to as a wet fry filter, allows for enhanced gas diffusion into the medium. Sensors, for example, an EC probe 119, are placed in the medium to monitor process parameters. Excess medium overflows to the nitrification unit through the line 120. The line 120 extends into a processing unit 121, such as that disclosed for apparatus 100a above, which, in turn, connects to the airlift pump 110, in accordance with the apparatus 100a, as detailed above.

FIG. 1E is an alternate system 100c which uses one or more tubes 184, which function as photobioreactors, and, for example, collectively function similar to the tank/photobioreactors 116, 116′ of the systems 100a, 100b, as detailed above, in which cyanobacteria is grown. The tubes 184 are, for example, made of translucent or transparent polyvinyl chloride (PVC) or PMMA, glass or other materials, which allow light transmission into the tubes 184. The tubes 184 are connected together by lines 186, and are fixed on a construct 188, in either a horizontal, vertical or another geometric setting.

Cyanobacteria are grown inside the tubes 184 in suspension or on carriers. Fresh medium, enriched with dissolved CO₂ and nitrogen, enters the tubes 184 from a line 114, and medium rich with dissolved ammonia and ammonium ions exits the tubes 184 through the line 120. An optional gas separator (gas outlet) 190 allows excess oxygen that is generated by the photosynthetic cyanobacteria to leave the system. Sensors, for example, an EC probe 119, are inserted into one or more of the tubes 184 to monitor process parameters. The line 120 extends into a processing unit 121, such as that disclosed for apparatus 100a above, which, in turn, connects to the airlift pump 110, in accordance with the apparatus 100a, as detailed above.

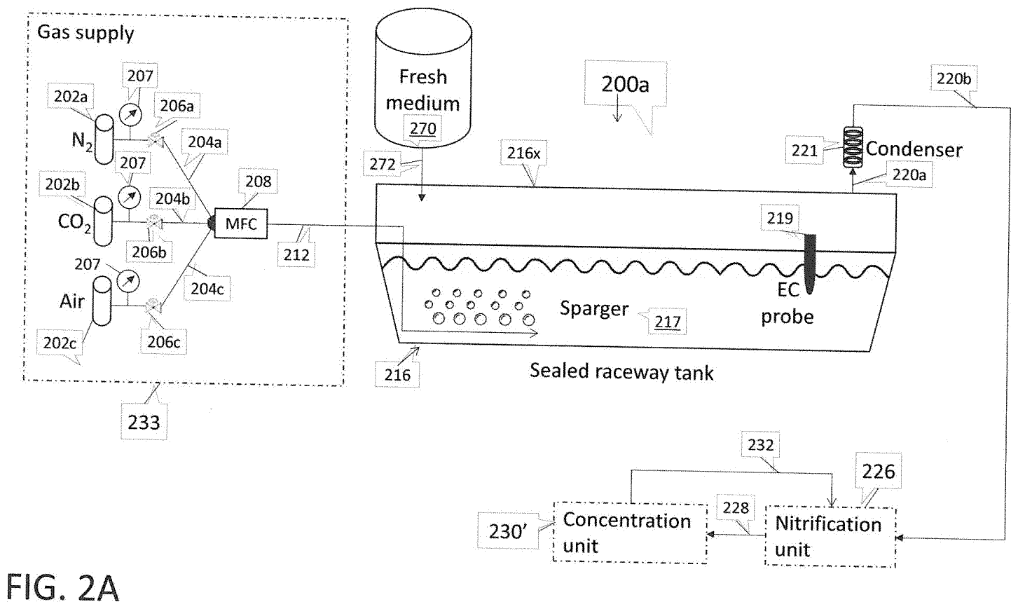

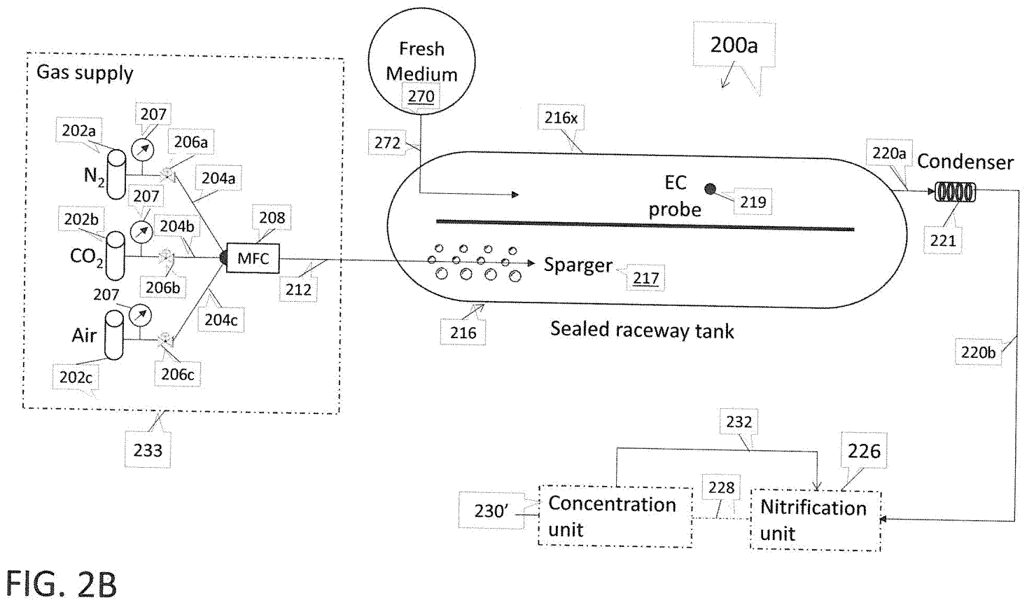

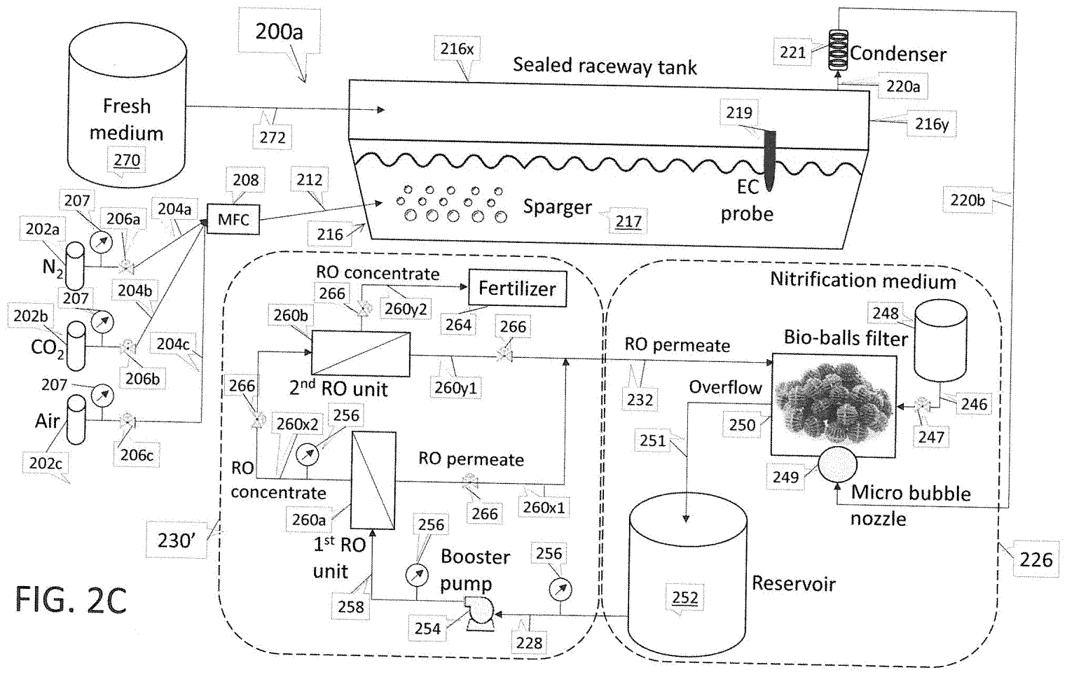

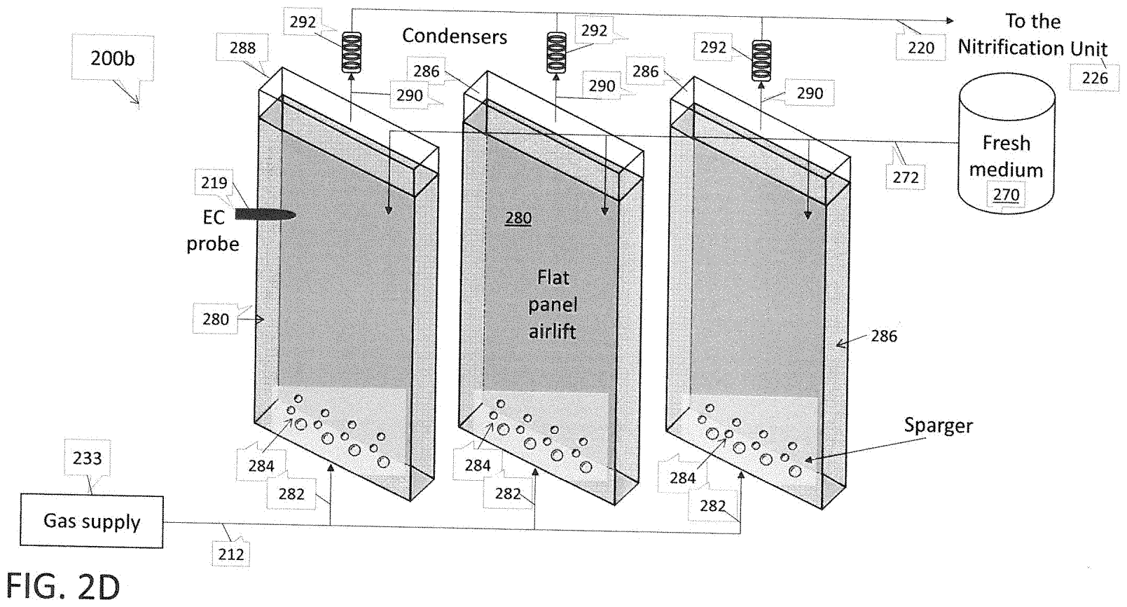

FIGS. 2A and 2B provide a system 200a for performing a process in accordance with another embodiment of the invention. The system 200a is similar in components (elements) to the system 100a, with the same or similar components to those shown in FIGS. 1A-1C and described above having the corresponding element number in the “200s”. These same or similar components are in accordance with the corresponding component (elements) descriptions above. Components of the system 200a, different from components of the system 100a, shown in FIGS. 2A-2C, are detailed below.

Sources of Nitrogen (N₂) gas 202a, Carbon Dioxide (CO₂) gas 202b and air 202c connect over lines 204a, 204b, 204c (with valves 206a, 206b, 206c), to form a gas stream, which is provided to a mass flow controller (MFC) 208. Pressure gauges 207, which are optional, extend along the lines 204a-204c. A line 212 extends from the MFC 208 to the tank 216. The MFC 208 flow rate controls circulation in the tank 216, by controlling gas influx in order to maintain a constant flow rate into the tank 216.

The tank 216 is an enclosed tank, covered by a cover 216x. The cover 216x is, for example, a transparent sheet or cover, made of materials such as polyethylene, polycarbonate, poly (methyl methacrylate) or glass. The cover 216x, for example, is such that it has at least one inlet and/or outlet airtight ports. The cover 216x is sealed to avoid loss of gas. Inlet and outlet are allowed only through the dedicated airtight ports.

Within the tank 216 is a partition 216p, a sparger 217 and an EC probe 219. The gas mixture, which was sparged into the covered and sealed tank 216 and builds up a positive pressure. In this enclosed tank 216, the cyanobacteria is grown at a high pH, around pH 9-10, so that the equilibrium between ammonia and ammonium favors the ammonia (NH₃) (at pH 9.25 the ratio is 1:1) Ammonia leaves the medium to the gas phase according to Henry's law, and then exits through the gas outlet ports into the condenser 221. The gas outlet is also enriched with Oxygen (O₂), which is a product of the photosynthesis performed by the cyanobacteria.

The tank 216, via a line 220a, connects to a condenser 221, for collecting water vapor. The condenser 221 liquefies water vapor, such that it returns to the tank. The gases, which have not condensed, e.g., ammonia rich gases, flow, via a line 220b, into a nitrification unit 226, and then through a line 228, to a concentration unit 230′. The condenser is optional and can be dispensed with in case a considerable amount of ammonia condensates as well.

FIG. 2C shows the system 200a in detail, on which an example operation is now described. Initially, cyanobacteria has been grown in suspension or on carriers in the tank 216 and released ammonium ions (NH₄⁺) into the water. This is due to the cyanobacteria fixing nitrogen, causing it to excrete ammonium into the water of the tank 216, as described above for the system 100a. In high pH conditions, for example, over a pH of 9-10, some of the ammonium is present as dissolved ammonia, and some of the ammonia escapes to the gas phase. In this system 200a (as well as system 200b) ammonium ions (NH₄⁺) are in the minority and ammonia, typically in the form of a soluble gas (ammonia gas), is in the majority. The ammonia gas has a high vapor pressure, allowing it to evaporate into the gas phase, such that the ammonia gas is bubbled into the nitrification unit 226.

Nitrogen (N₂), Carbon Dioxide (CO₂), and/or air, from sources 202a-202c, are injected through the MFC 208, into the enclosed tank 216, by a sparger 217. The sparging encourages the expulsion of ammonia from the solution into the gas phase in the headspace 216y. The tank 216 has previously or contemporaneously been filled with fresh medium, as detailed herein, from a source 270, through a line 272.

The water vapor, rich with ammonia (NH₃) and Oxygen (O₂), from the enclosed tank 216, flows into the condenser 221. The ammonia rich gas flows into the nitrification unit 226. The nitrification unit 226 includes, for example, a micro bubble nozzle 249, a bio-filter 250, and a reservoir 252, connected by a line 251. The bio-filter 250 includes the filtration media of bio balls or other nitrifying bacteria and/or carriers therefor, as detailed for the filter (bio-filter) 150 above.

The ammonia and oxygen rich gas, enters the bio-filter 250 as small bubbles, by passing through a micro bubble nozzle 249, including an element such as an air stone, Venturi nozzle, micro/nano bubble diffuser or the like. The ammonia dissolves into the solution (e.g., a liquid) in the bio-filter 250, and is converted to Nitrate (NO₃⁻), in the solution. The excess oxygen in the gas influx supports the high oxygen demand of the nitrification process. The Nitrate rich solution (e.g., a liquid) is then received in the reservoir 252. The booster pump 254 forces the Nitrate rich solution into the concentration unit 230′. Top off nitrification medium 248, which resembles the fresh medium 270, is added to the bio-filter 250 where needed, through a line 246 controlled by a valve 247. Pressure gauges 256 are, for example, placed along the line 228 servicing the booster pump 254, as well as the lines 260x1, 260x2, 260y1, 260y2, in the concentration unit 230′.

The concentration unit 230′ includes reverse osmosis (RO) units, for example two RO units 260a, 260b, arranged sequentially (but can also be arranged in parallel), the booster pump 254, and a reservoir 264 for the concentrate from the RO units 260a, 260b.

The booster pump 254 pumps the Nitrate rich solution through the RO units 260a, 260b at a rate sufficient to separate the accumulated Nitrate rich solution into concentrate. The booster pump 254 pumps the Nitrate rich solution, via line 258, into the first RO Unit 260a. The permeate from the RO unit 260a is sent along line 260x1 which continues into line 232 to the bio-filter 250 of the nitrification unit 226. The concentrate from the RO unit 260a is sent along line 260x2 to the second RO unit 260b. The permeate from the second RO Unit 260b is sent along line 260y1 which continues into line 132 to bio-filter 250. The concentrate from the second RO Unit 260b is sent along line 260y2 to the reservoir 264, so as to be recovered as product, e.g., fertilizer (liquid fertilizer).

In the system 200a, as shown in FIG. 2C, in the concentration unit 230′ the lines 260x1, 260x2, 260y1 and 260y2 include valves 266. These valves 266, along with the valves 206a-206c, 247, sparger 217, EC probe 219, and booster pump 254, may be controlled manually, automatically by a computer control system, or combinations thereof. Also, the pressure gauges 207, 256 may also be connected to the computer control system.

FIG. 2D shows a system 200b, which additionally references the components of the system 200a, as presented, for example, in FIGS. 2A and 2B. A gas supply 233, via line 212, supplies gas (e.g., one or more of Nitrogen, Carbon Dioxide and/or Air) to flat panel airlift reactors 280, in which cyanobacteria are grown in suspension or on carriers. The system 200b uses one or more panels 280, which function as photobioreactors, and, for example, collectively function similarly to the tank/photobioreactor 216 of the system 200a, as detailed above, in which cyanobacteria are grown.

The panels 280 are made of translucent or transparent PMMA, polycarbonate, glass or another material, to allow light into the panels 280, and may have different degrees of compartmentalization in order to optimize gas diffusion into the medium, which fills each panel 280. The gas inlet 282, from the line 212, provides CO₂ and nitrogen for each panel 280, which is received in the respective panel 280 by entering into a sparger 284. The entering gas creates an airlift effect inside the panel 280, which aids in agitation and gas exchange.

The system 200b operates at high pH levels, around pH 9-10, that favors the conversion of ammonium ions fixed by the cyanobacteria into ammonia gas that accumulates in the headspace 286. Ammonia and oxygen rich gas leaves though the outlet 290 into a condenser 292, and then continues to the nitrification unit 226, and to the concentration unit 230′, through the line 220. Top off medium (from a storage or source 270) enters each panel 280 through a line 272. Sensors, for example, an EC probe 219, are inserted into the panels 280 to monitor process parameters.

Alternately, the systems 100a, 100b, 100c, 200a, 200b may include a water pump for the tanks or photobioreactors (bioreactors) 116, 116′, 216. This water pump may be used with the airlift pump 110, or in substitution thereof. The water pump drives fresh medium, water, and/or other substances, as detailed above, into the respective tank 116, 116′, 216, as well as driving flow out from the tank 116, 116′, 216.

The systems 100a, 100b, 100c, 200a, 200b are constructed to operate to continuously produce ammonia and to convert it into other forms of fixed nitrogen, such as Nitrate and nitrate based products, for use as a raw product, such as a fertilizer, e.g., liquid fertilizer, in agriculture. This continuous operation is continuous for time periods, for example, of weeks, months, and even years.

The systems 100a, 100b, 100c may also use a top off pump for the tank (photobioreactor or bioreactor) 116, 116′, which is controlled by a level probe. The top off pump delivers fresh medium to the tank 116, 116′. The fresh medium is comprised of, but not limited to: water, compensating losses due to evaporation, micro nutrients for consumption by the cyanobacteria, and acid for reducing extra alkalinity formed during ammonium evolution. For example, the top off pump is controlled by gravity or by a physical apparatus.

Alternately, in the systems 100a, 100b, 100c, 200a, 200b, limited base is added to the tank (photobioreactor) 116, 116′, 216, promoting an alkaline outlet. The base input is adjusted to compensate the natural acidification in the nitrification unit, and to achieve optimal pH for both the cyanobacteria and the nitrifying bacteria.

In other embodiments, the particulate filter 122 includes a cross flow ultra-filter to recover cyanobacteria and return them back to the main tank. Some of the cells may be discarded in order to maintain a dilution rate and the cell density in the tank. A cross flow nano-filter may be used to recover macro molecules, such as the ammonium uptake inhibitor (MSX), and to return it back to the main tank. Example for cross flow filters include, Iris 3038 (Polyacrylonitrile (PAN), 40 kDa cut off) ultra-filtration membrane, available from Rhodia-Orelis of Miribel, France, and Nano-filtration Membrane Model NFX (Polyamide, 100 Da cut off) available from Synder Membrane Technology Co. (Snyder Filtration), Vacaville, Calif., USA.

In other embodiments, a settling chamber is included or added in the outlet area of the tank by using one or more baffles, partitions, basins or another method that exploits gravity to settle the cyanobacteria and separate them from the outlet solution. The sediment may be removed through another outlet. Micro-carriers such as clay minerals may be used to immobilize the cells in aggregates with a larger density and a faster settling time. Macro-carriers such as Fibra-Cel disks (Eppendorf, Germany) may be used to enable simple separation of the cells from the medium. In these embodiments, the outlet contains mostly cell free media and the cross flow filter may be replaced with a simpler particulate filter.

In other embodiments, cationic media such as, but not limited to, carbonates or alkalis of calcium, magnesium or potassium, is added to the nitrification unit 126, 226, for example, with, or instead of, the bio-filters 150, 250, in order to balance the drop in pH during the nitrification process, and to stabilize the Nitrate as a solubilized salt of one of the cations mentioned above as examples.

In other embodiments of the systems 100a, 100b, 100c, 200a, 200b, Nitrate rich solution from the nitrification unit 126, 226, is collected in the reservoir 152, 252 during light hours of the day, and is then concentrated using the RO units 160a, 160b, 260a, 260b, that continue to run during dark hours of the day as well. The reservoir 152, 252 promotes a more effective process by employing RO units 160a, 160b, 260a, 260b with less capacity. The permeate is returned to the tanks (photobioreactors) 116, 116′, 216 or to the nitrification unit 226 (via line 232) as pure water top off supplement, or is mixed with fresh medium for the same purpose.

In other embodiments of the systems 100a, 100b, 100c, 200a, 200b, some of the concentrate is returned to the reservoir to pass again through the RO units 160a, 160b, 260a, 260b in order to achieve a higher concentration of the final product.

Both systems 100a, 100b, 100c, 200a, 200b may be such that physical and chemical conditions inside the tanks (photobioreactors) 116, 116′, 216, reservoirs 150, 250 and vessels 164, 264, are controlled by probes for pH, temperature, dissolved oxygen, ammonia, CO₂, turbidity, conductivity, oxidation reduction potential or any other process parameter. The conditions are then regulated using the proper intervention such as acid/base pumps, temperature control units, gases flow rates or circulation rate, in a manner that is practiced amongst those skilled in the art.

The systems 100a, 100b, 100c, 200a, 200b present several design concepts for optimized cyanobacteria growth. The systems 100a, 100b, 100c, 200a, 200b operate in either an open or a sealed setting. For example, the embodiments of the raceway tank (bioreactor) 116, 216, the wet dry reactor (bioreactor) 116′, the tubes 184, and the flat panel airlift 280, can be used in an open or sealed setting.

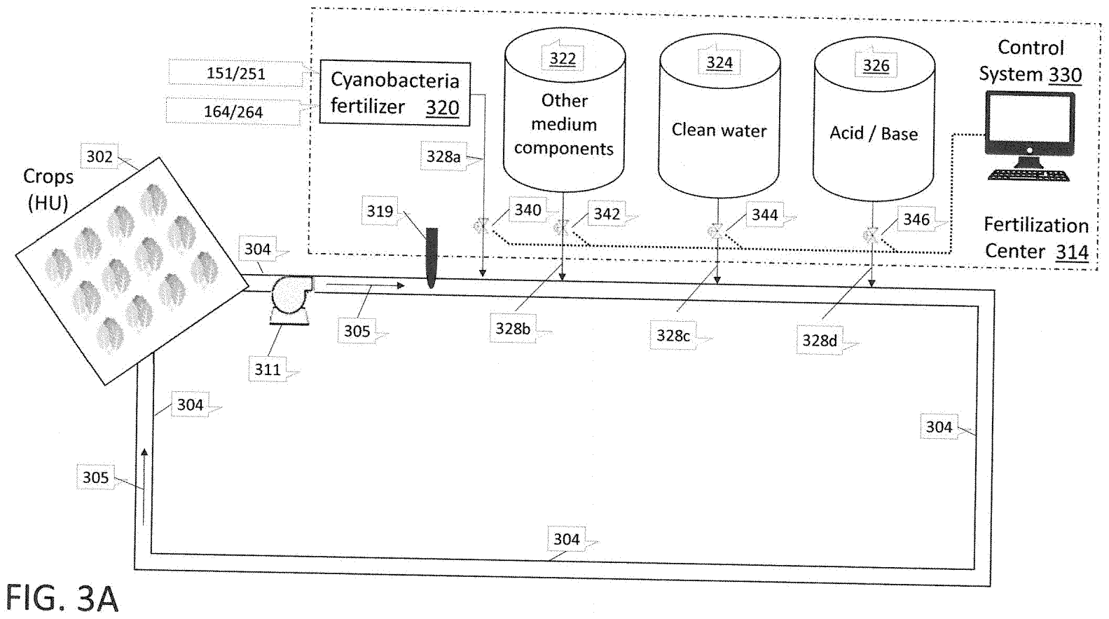

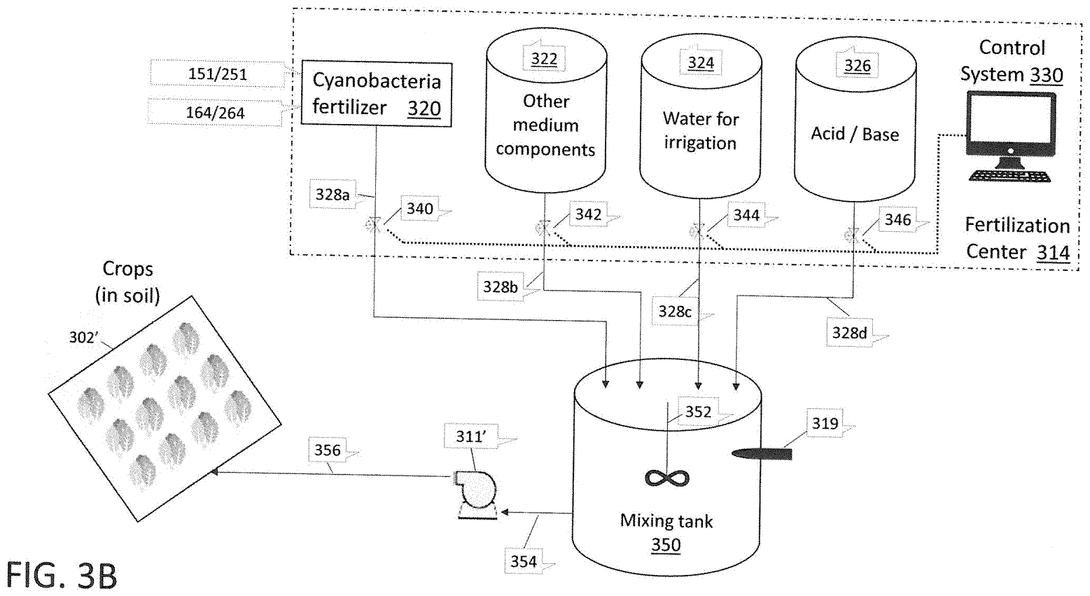

FIG. 3A provides a system for a continuous fertilization of crops that are grown for example in a hydroponic unit (HU) 302. The crops are fertilized along line 304 that defines a circulation path (in the direction of the arrow 305) with the nitrate rich product of the cyanobacteria system 100a, 100b, 100c, 200a, 200b, as detailed above, which is, for example, fertilizer (e.g., cyanobacteria fertilizer). The fertilizer may be, for example, dilute from system 100a, 100b, 200a, 200b components 151/251 or concentrated from the systems 100c, 200b, elements 164/264, and is, for example, continuously produced by a proximate cyanobacteria system, such as those 100a, 100b, 100c, 200a, 200b, detailed above. The cyanobacteria fertilizer, for example, is fortified with other medium elements, such as phosphorous, iron and trace elements. These elements may, or may not be certified as inputs for organic agriculture, for example, potash, rock phosphate, and various sulfates.

Water from a hydroponic unit (HU) 302, holding crops or other vegetation, is pumped, by a pump 311, through a line 304 to a fertilization center 314, where it is enriched by such elements as the cyanobacteria fertilizer 320, other medium components 322, clean water 324, acid/base 326 or others, through lines 328a, 328b, 328c and 328d respectively. The water parameters in the circulating water, along the circulation path 304 are monitored by, for example an EC probe 319 (similar to EC probes 119, 219 as described above), or other sensors in the line 304 or in the cyanobacteria fertilizer supply 320, for measuring parameters: EC, pH, temperature, dissolved oxygen, ammonium and nitrate levels, redox potential or the like.

The sensor data is analyzed by a processor-based (computerized) control system 330, which is programmed to sense proper levels of fertilizer, medium components, clean water, acid/base, and adjust the levels thereof in the circulation path by controlling valves 340, 342, 344, 346 for the cyanobacteria fertilizer 320, other medium components 322 (e.g., decontamination agents such as chlorine dioxide), clean water 324 (e.g., reverse osmosis water), acid/base 326 (e.g., concentrated acid or base to adjust pH), respectively. The processor based control system 330 may also control the cyanobacteria fertilizer 320, other medium components, clean water 324, acid/base 326, respectively via dosing pumps, or other regulating apparatus. The fertilized and treated water is pumped back to the crops though line 304. In case the crops are grown in soil using fertigation or another non-hydroponic growing method, line 304 functions as a one way line from the irrigation water components 320, 322, 324, 326 to the crops 302. An optional mixing tank may be included to mix these components before they are pumped to the crops.

FIG. 3B shows another system for crop fertilization. In FIG. 3B, identical or similar components have the same element numbers as those in the system of FIG. 3A. In this system of crop fertilization, the fertilization center 314, feeds into a mixing tank 350. Accordingly, fertilizer 320, medium components 322, irrigating water 324 and Acid/Base, via lines 328a-328d, is fed to the mixing tank 350, where the components are mixed or agitated by a stirrer (mixer or agitator) 352 or the like.

The liquid fertilizer in the mixing tank 352 travels over a line 354, where it is pumped by a pump 311′ (similar to pump 311 as detailed above) through a line 356 to crops in soil 302′.

Although the invention has been described in conjunction with embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for producing ammonia comprising:
growing nitrogen fixing cyanobacteria in a bioreactor;
exposing the cyanobacteria, while in a live state, to an inhibitor, in the bioreactor, such that the inhibitor induces the cyanobacteria to release ammonia; and,
preserving the cyanobacteria in the live state for continuously producing the ammonia.

2. The method of claim 1, additionally comprising:
providing media to the bioreactor.

3. (canceled)

4. The method of claim 1, wherein the ammonia includes at least one of ammonia, ammonium ions, or, a mixture of ammonia and ammonium ions.

5. The method of claim 4, additionally comprising, controlling the pH level in the bioreactor to alter the balance of ammonia to ammonium ions.

6. (canceled)

7. The method of claim 1, wherein the bioreactor includes liquid solution.

8.-9. (canceled)

10. The method of claim 1, wherein the cyanobacteria is immobilized on one or more carriers, including one or more of: alginate, carrageenan, glass beads, polyvinyl, polyester, or polyurethane foams, polyester fibers, cellulosic or poly-sulfone hollow fibers, or, clay particles.

11.-13. (canceled)

14. The method of claim 1, wherein the cyanobacteria is from the family Nostocaceae.

15.-18. (canceled)

19. The method of claim 1, wherein the bioreactor includes a tank.

20.-23. (canceled)

24. The method of claim 1, wherein the bioreactor includes a sparger.

25. The method of claim 4, wherein the ammonia includes ammonia gas dissolved in the liquid solution as a mixture of soluble ammonia gas and ammonium ions.

26. The method of claim 25, wherein: 1) the ammonia gas dissolved in the liquid solution is exposed to nitrifying bacteria to produce a Nitrate based product; or; 2) the ammonia gas and ammonia ions are exposed to nitrifying bacteria to produce a Nitrate based product.

27.-29. (canceled)

30. The method of claim 1, wherein the cyanobacteria is grown at an alkaline pH.

31. The method of claim 30, wherein the pH is approximately 9 to 10.

32.-36. (canceled)

37. The method of claim 2, wherein the inhibitor includes at least one of: MSX (L-methionine-DL-sulfoximine), MSO (L-methionine-sulfone), phosphinothricin ((RS)-2-Amino-4-(hydroxy(methyl)phosphonoyl)butanoic acid), or, Bialaphos (L-Alanyl-L-alanyl-phosphinothricin) or Glyphosate (N-(phosphonomethyl)glycine).

38. (canceled)

39. A method for producing ammonia comprising:
growing nitrogen fixing cyanobacteria in a bioreactor, wherein the cyanobacteria is a mutant strain of cyanobacteria;
controlling the environment in the bioreactor, such that the cyanobacteria, while in a viable state, releases ammonia;
preserving the cyanobacteria in the viable state for continuously producing the ammonia; and,
extracting the ammonia from the bioreactor including separating the ammonia from the cyanobacteria and the inhibitor.

40. The method of claim 39, where the viable state includes a live state and, the controlling the environment includes controlling one or more of agitation, temperature, and pH in the bioreactor.

41. (canceled)

42. The method of claim 39, where the mutant strain of cyanobacteria includes at least one of: A. variabilis, or, A. siamensis.

43.-44. (canceled)

45. A method for producing Nitrate based products from ammonia comprising:
growing nitrogen fixing cyanobacteria in a bioreactor;
exposing the cyanobacteria, while in a viable state, to an inhibitor, in the bioreactor, such that the inhibitor induces the cyanobacteria to release ammonia;
preserving the cyanobacteria in the viable state for continuously producing the ammonia; and,
exposing the ammonia to nitrifying bacteria to produce a Nitrate based product, or, extracting the ammonia in at least a gas and/or a liquid phase from the bioreactor including separating the ammonia from the cyanobacteria and the inhibitor.

46. The method of claim 45, wherein the Nitrate based product includes fertilizer.

47. (canceled)
